(19) United States
(12) Patent Application Publication
(10) Pub. No.: US 2007/0219625 A1
(43) Pub. Date: Sep. 20, 2007

(54) Assembly for the Treatment of Bifurcations

(75) Inventors: Andrea Venturelli, Concesio (IT); Paolo Rezzaghi, Soave di Porto Mantovano (IT)

Correspondence Address: SHOEMAKER AND MATTARE, LTD, 10 POST OFFICE ROAD - SUITE 110, SILVER SPRING, MD, 20910, US

(73) Assignee: INVATEC S.R.L., Roncadelle, Brescia (IT)

(21) Appl. No.: 11/575,950

(22) PCT Filed: Sep. 29, 2004
(86) PCT No.: PCT/IT04/00520

§ 371 Date: Jun. 4, 2007

(51) Int. Cl. A61F 2/06

(52) U.S. Cl. 623/1.16

(57) ABSTRACT

A stent for the endoluminal treatment of stenosis at a vessel bifurcation comprises at least two cylindrical portions extending along different axes. A catheter and a mandrel for placing the stent are described as well.

The present invention relates to an assembly intended for endoluminal treatment of those blood vessel bifurcations affected by stenosis.

Particularly, the invention relates to an assembly intended for the endoluminal treatment of bifurcations with stenosis limited to one branch, typically the side branch.

Stents are known to be used for the endoluminal treatment of blood vessels affected by stenosis. The vessel inner diameter, which is pathologically narrowed by the presence of stenosis, is dilated by carrying out an angioplasty operation by means of a catheter. By using the stent, the vessel wall can be supported and kept dilated such as to prevent the inner diameter from narrowing back after the angioplasty operation.

In the particular field of bifurcation treatment, the traditional stents of cylindrical shape suffer from the drawback that they do not provide a suitable support to all bifurcation areas.

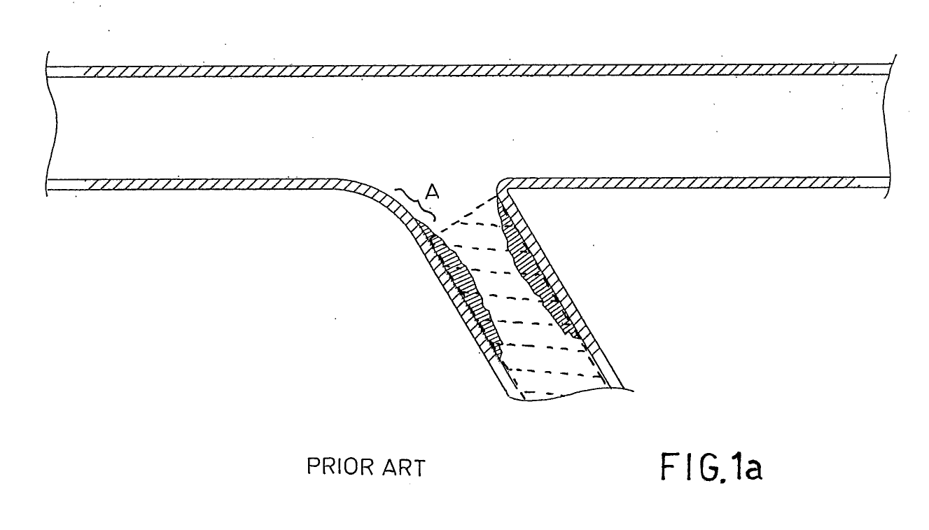

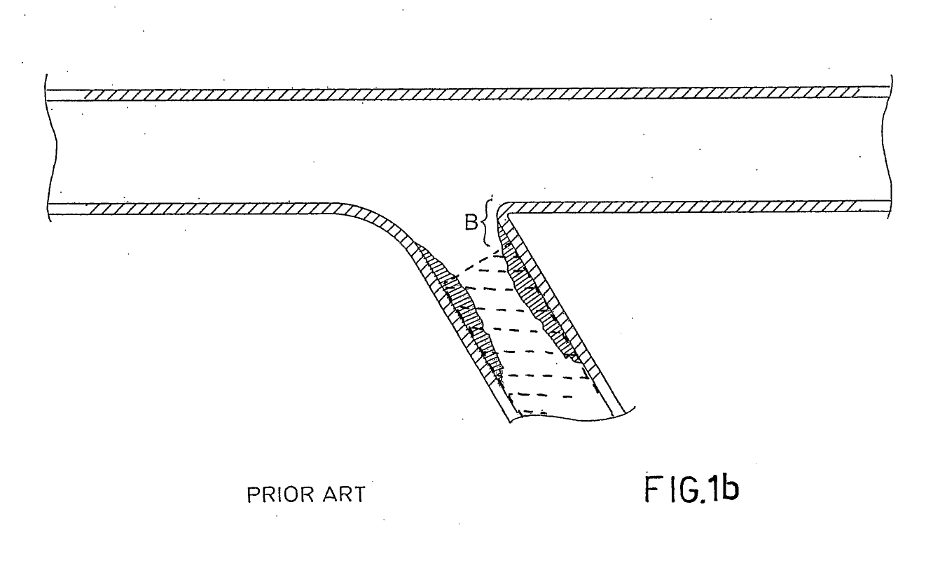

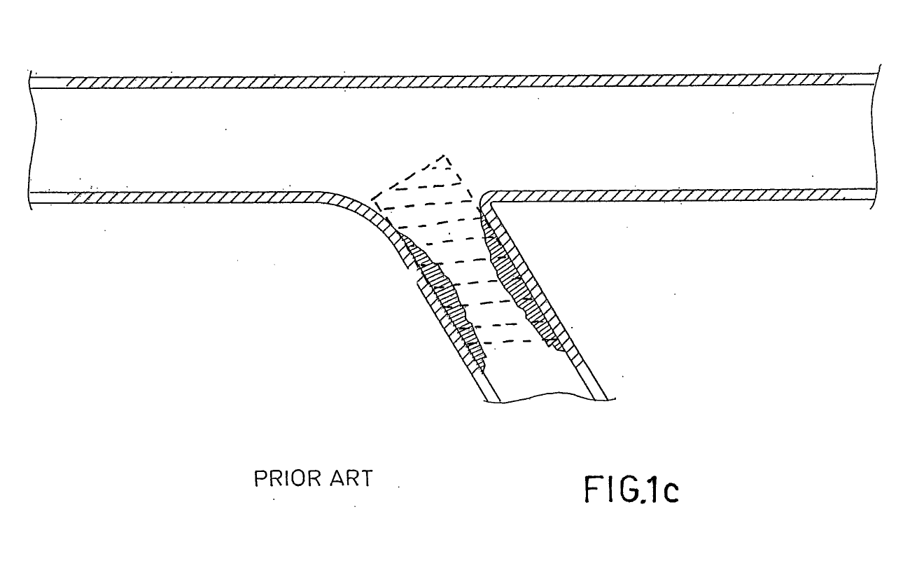

By defining a main branch and a side branch within the bifurcation, if the stenosis is located only within the side branch, placing a stent within the main branch is not required. In this case, the most proximal area of the side branch (see for example the area indicated with A in the annexed FIG. 1a) is devoided of any support, since the traditional stent being placed in the side branch will not cover it.

Similarly, the so-called carina, i.e. the bifurcation area (see for example the area indicated with B in the annexed FIG. 1b), may lack suitable support since the accuracy in placing the stent is only ensured by the operator's skill. Neither the traditional stents, nor the catheter employed for placing them offer the possibility of checking the location relative to the carina. Therefore, the case may occur that, wishing to prevent the stent from interfering in the blood stream of the main branch after it has been positioned (such as in the example in FIG. 1c), the operator will tend to place it slightly deeper within the side branch.

The object of the present invention is to conceive and provide a catheter and a stent allowing to overcome the drawbacks mentioned above with reference to the prior art.

Particularly, the task of the present invention is to provide a stent capable of providing a suitable support both to the part most proximal of the lateral side and the bifurcation carina. Furthermore, the task of the present invention is to provide a catheter assembly allowing to accurately check the location of the stent upon implantation.

This object and this task are achieved by means of a stent, catheters and a catheter assembly, respectively, in accordance with claims 1, 10, 22 and 30.

Further characteristics and advantages of the invention will appear from the description given below of preferred embodiments, which are intended to be indicative and non-limiting examples, with reference to the annexed figures, in which:

FIGS. 1a, 1b and 1c schematically illustrate the placement of a stent according to the prior art in a bifurcation affected by stenosis in the side branch, in dotted line;

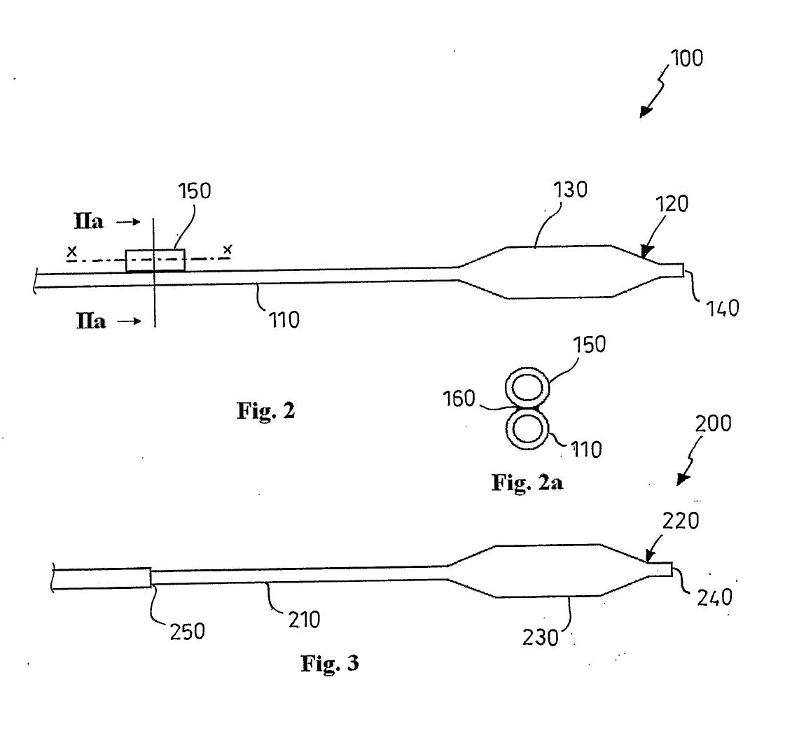

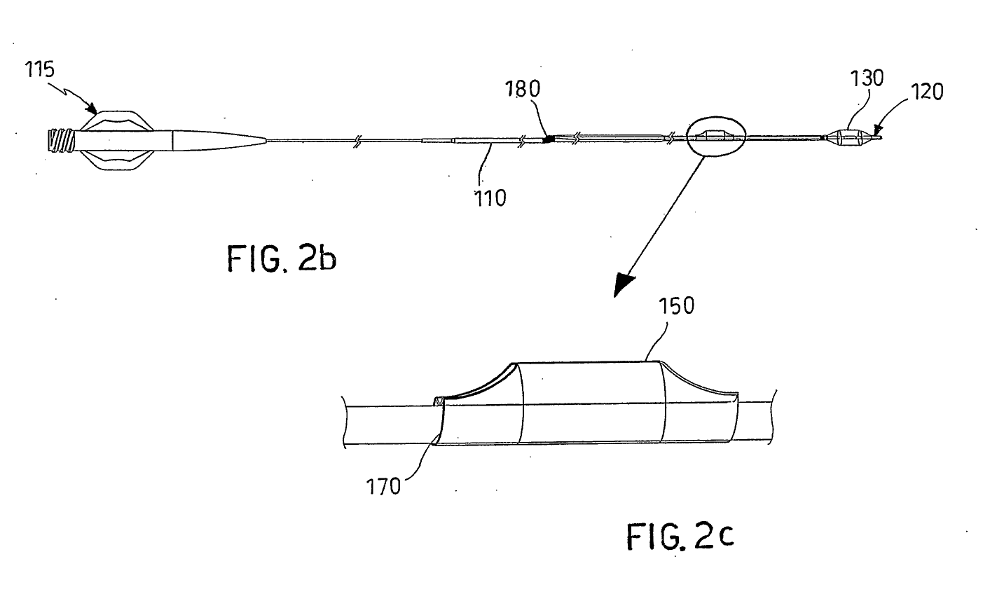

FIG. 2 schematically illustrate a first embodiment of a first catheter according to the invention;

FIG. 2a schematically illustrates a section taken along the line IIa from FIG. 2;

FIG. 2b schematically illustrates a second embodiment of a first catheter according to the invention;

FIG. 2c illustrates a detail of the catheter from FIG. 2b;

FIG. 3 schematically illustrates an embodiment of a second catheter according to the invention;

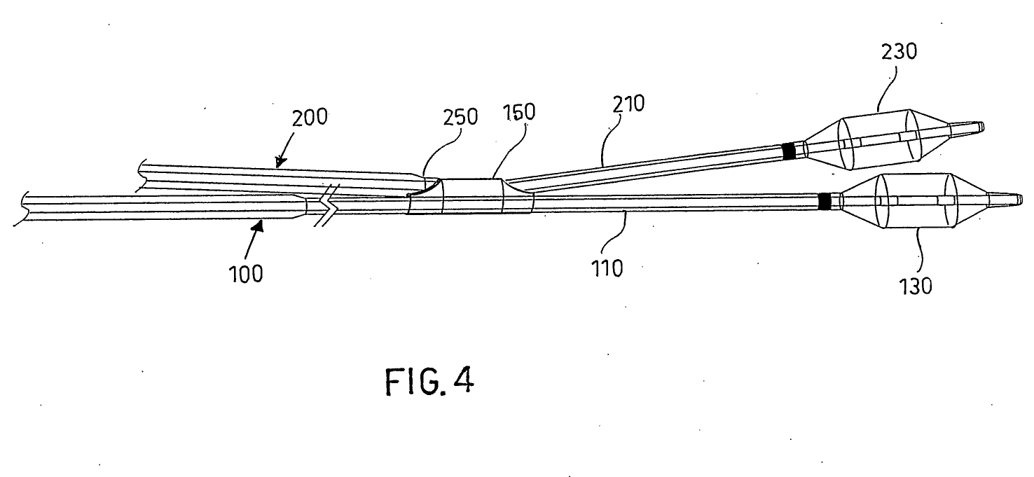

FIG. 4 schematically illustrates an assembly comprising the catheters from FIGS. 2 and 3 according to the invention;

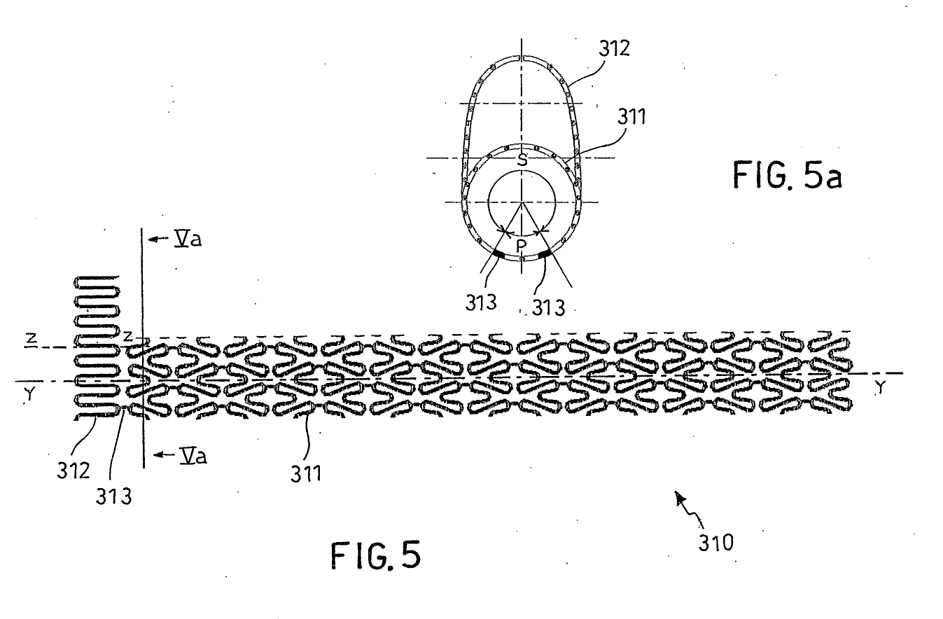

FIG. 5 schematically illustrates a first embodiment of a stent according to the invention;

FIG. 5a illustrates a section taken along the line Va from FIG. 5;

FIG. 6 illustrates an assembly according to the invention comprising the catheters from FIG. 2 and the stent from FIG. 5;

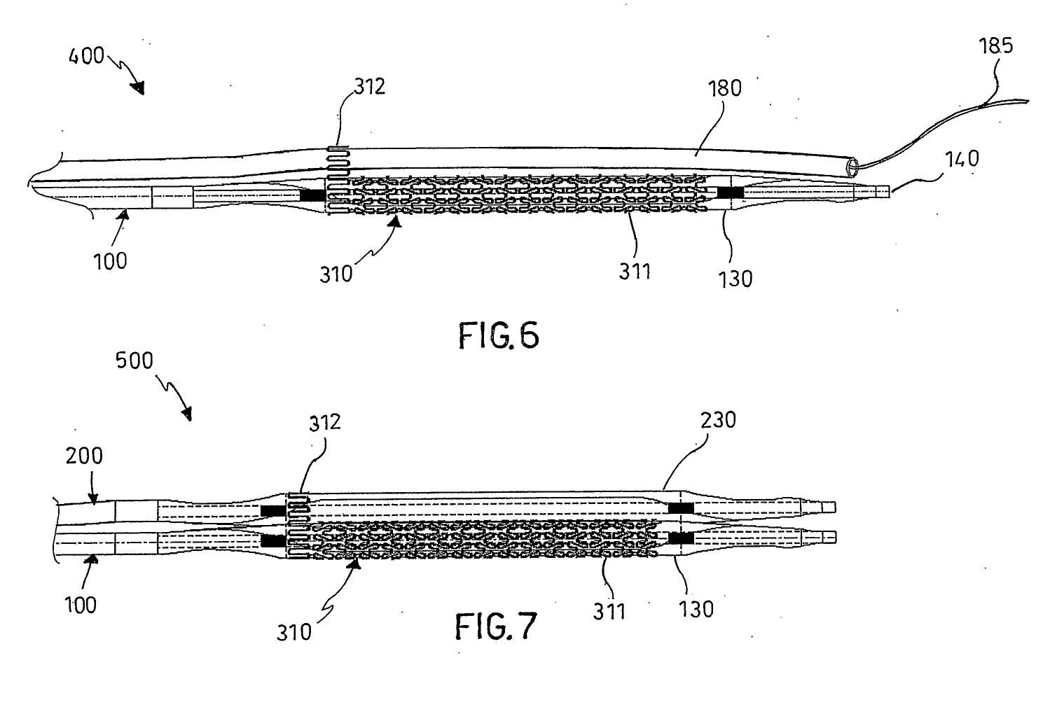

FIG. 7 illustrates an assembly according to the invention comprising the catheters from FIGS. 2 and 3 and the stent from FIG. 5;

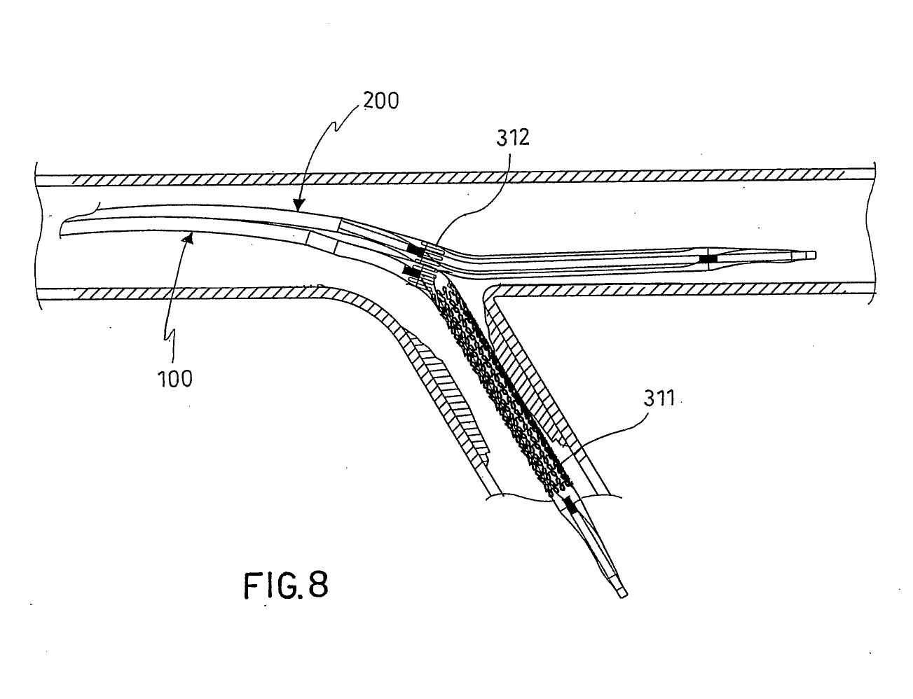

FIG. 8 illustrates the assembly from FIG. 7 in a first step of use within a bifurcation;

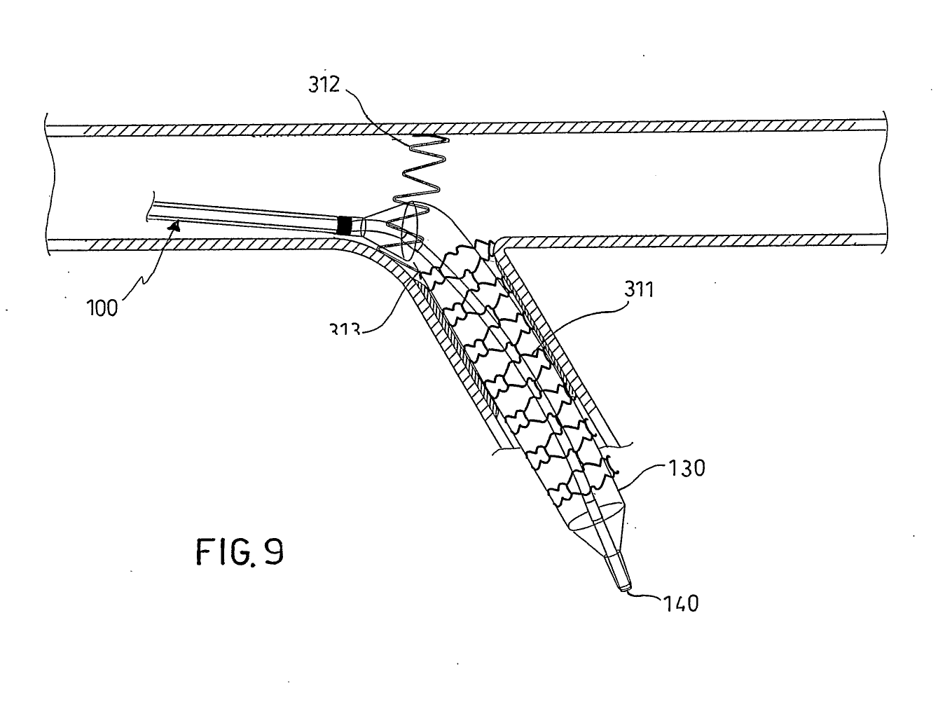

FIG. 9 illustrates the assembly from FIG. 7 in a second step of use within a bifurcation;

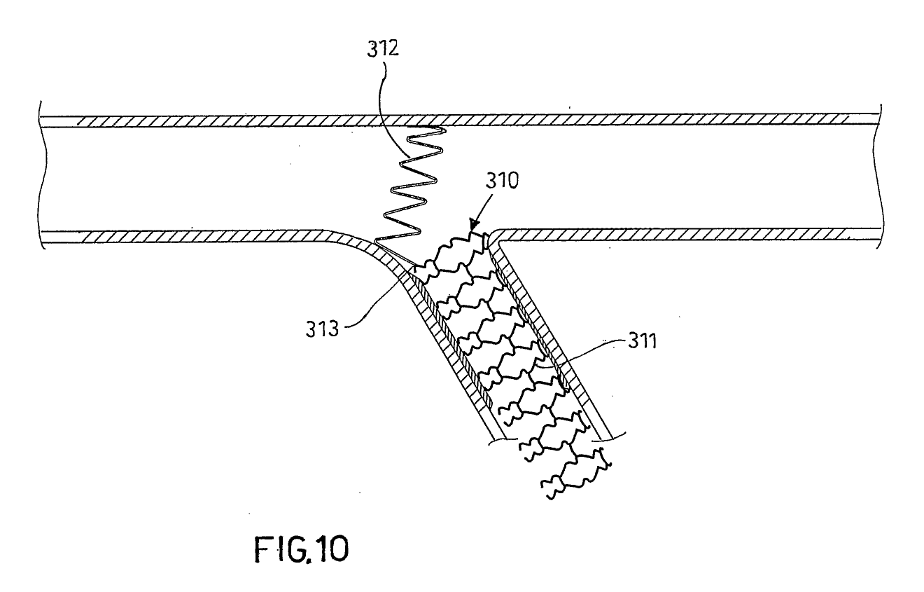

FIG. 10 illustrates the stent from FIG. 5 when placed in a bifurcation;

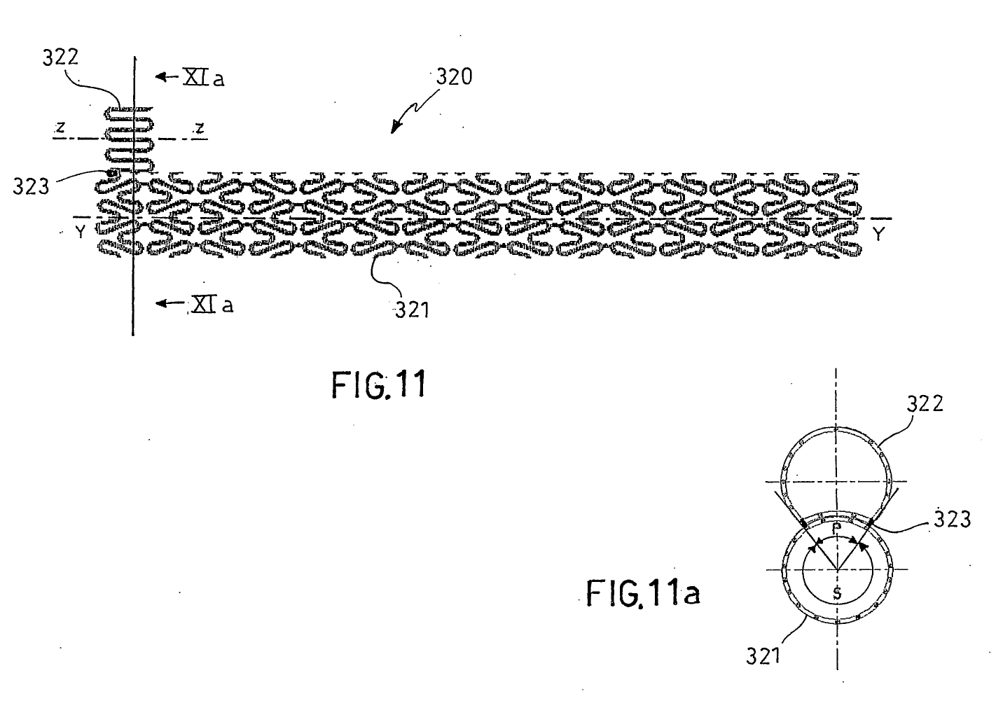

FIG. 11 illustrates a second embodiment of a stent according to the invention;

FIG. 11a illustrates a section taken along the line XIa from FIG. 11;

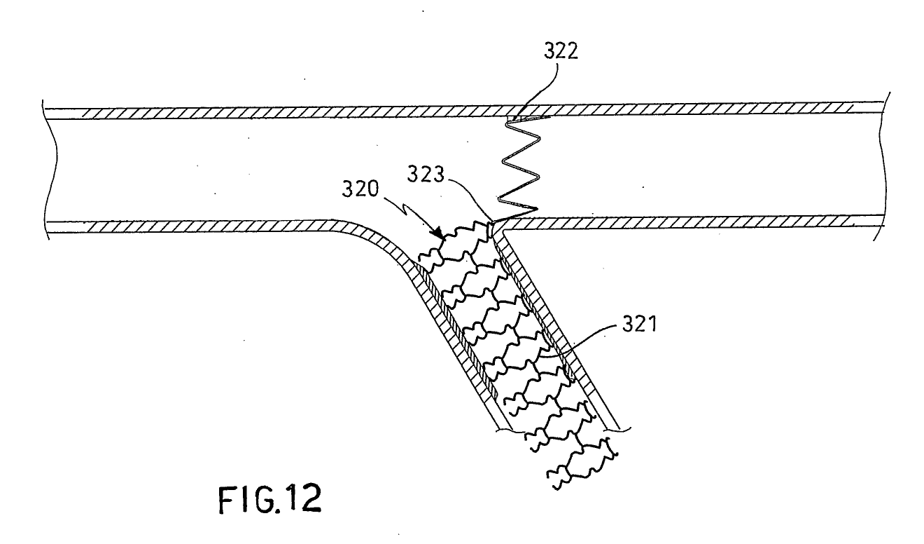

FIG. 12 illustrates the stent from FIG. 11 when placed in a bifurcation;

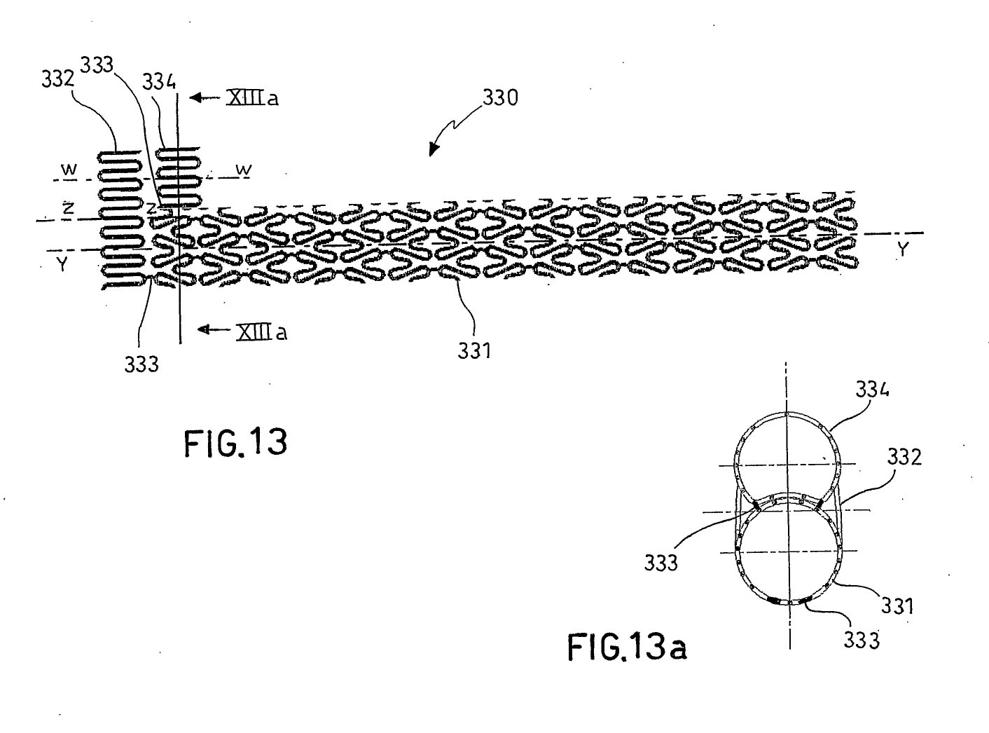

FIG. 13 illustrates a third embodiment of a stent according to the invention;

FIG. 13a illustrates a section taken along the line XIIIa from FIG. 13;

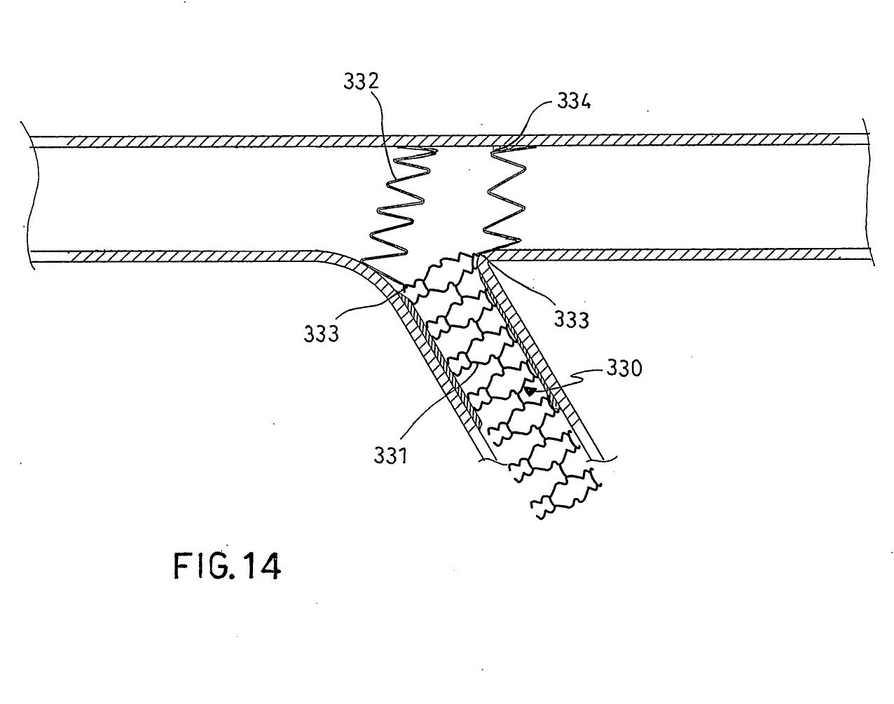

FIG. 14 illustrates the stent from FIG. 13 when placed in a bifurcation;

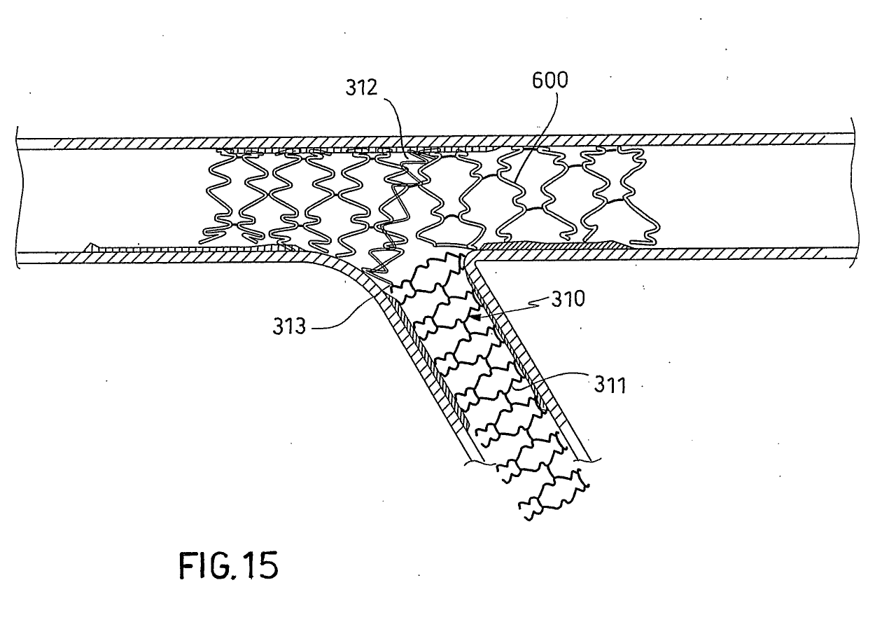

FIG. 15 illustrates the stent from FIG. 5 when placed in a bifurcation together with a stent of the known type.

With reference to the above figures, with 100 has been indicated a first catheter as a whole. The first catheter 100 comprises a tubular body 110, known per se, having a proximal end 115 and a distal end 120, known per se. The distal end 120, in turn, comprises a balloon 130 for angioplasty and a distal port for a guide wire 140, as is known in the art.

The first catheter 100 further comprises an eyelet 150 being arranged laterally to the tubular body 110, and integral therewith.

In accordance with a preferred embodiment of the first, catheter 100 according to the invention, the eyelet 150 defines an x-x axis which is locally parallel to the tubular body 110 of catheter.

In accordance with an embodiment, the eyelet comprises a substantially unextensible thread slot.

In accordance with a preferred embodiment, the eyelet 150 comprises a tube length being structurally fastened to the tubular body 110 such as to be integral therewith.

The structural fastening between the tubular body 110 and the eyelet 150 can comprise for example a gluing or welding 160 such as in the example from FIG. 2a. The structural fastening can also comprise a sheath 170 simultaneously enveloping the tubular body 110 and the eyelet 150, such as shown in the example from FIG. 2c. Alternatively, the eyelet 150 and the tubular body placed beside it can be made as one piece by extrusion. Finally, the structural fastening between the tubular body 110 and the eyelet 150 can also comprise any other element which is deemed suitable to ensure a firm fastening of the catheter in any usage condition.

The length of the tube comprised within the eyelet 150 for example can be cut along a perpendicular plane relative to x-x axis, such as in the example from FIG. 2. The length of tube can also be cut along a curved and biased surface relative to the x-x axis, such as in the example from FIG. 2c.

The eyelet 150 is placed at a preset distance from the distal end 120 and particularly the balloon 130.

The first catheter 100 comprises a proximal port for the guide wire, which is known per se. In accordance with a preferred embodiment, this proximal port 180 is placed proximal of the eyelet 150.

With 200 has been indicated a second catheter according to the invention as a whole. The second catheter 200 comprises a tubular body 210 known per se having a proximal end and a distal end 220, which are known per se. The distal end 220 comprises in turn an angioplasty balloon 230 and a distal port for a guide wire 240, as is known in the art.

The second catheter 200 further comprises a shoulder 250 being arranged on the tubular body 210. The shoulder 250 comprises an abrupt variation in the outer diameter of tubular body 210. The variation is arranged such that the immediately distal diameter relative to the shoulder 250 is smaller than the outer diameter such as defined by the shoulder itself.

The shoulder 250 can comprise a step perpendicular to the outer surface of tubular body 210 as in the example from FIG. 3, or rather may comprise a tapering joining the shoulder maximum diameter to the tubular body diameter, such as in the example in FIG. 4.

With reference to FIGS. 5 to 10, with 310 is indicated a first embodiment of a stent according to the invention as a whole.

The stent 310 comprises a first cylindrical portion 311 and a second cylindrical portion 312. The cross-section of the cylindrical portions 311 and 312, in accordance with the geometric definition of cylinder in the broadest meaning of the word, either elliptic or any other closed loop which may be suitable for the particular use of a stent 310.

The first portion 311 and the second portion 312 are structurally connected to each other by means of at least two bridges 313. The bridges 313 engage a first length, along the closed loops being defined by the cross section of the stent. For example, the first length of the closed loop being defined by the first portion 311, engaged by the bridges 313, is indicated with P in FIG. 5a. A second length of the closed loop is thus also defined, which is complementary to the first one and is not interested by the presence of the bridges 313. This second length is indicated with S in FIG. 5a.

In the stent 310 according to the invention, the first length P is shorter than second length S. In other words, the structural connection between both portions 311 and 312 of the stent 310 comprises a plurality of bridges 313 being unevenly distributed over the closed loop being defined by the cross-section of the portions.

In accordance with an embodiment, the first length P is shorter than the half, preferably one third and still more preferably shorter than one fourth of the length of second length S.

The y-y axis of the first portion 311 and the z-z axis of the second portion 312 are distinct. Furthermore, the closed loop defined by the cross-section of the first portion 311 is at last partially comprised in the closed loop being defined by the cross-section of the second portion 312. In other words, the proximal end of the first portion 311 is connected to the distal end of the second portion 312 by bridges 313.

In accordance with an embodiment, the bridges 313 have different lengths and/or elasticity from one another. The bridges being closer to the first length P of the closet loop are required to have a smaller length and/or elasticity than the bridges arranged proximal of the ends of the first length P.

In accordance with one embodiment of the stent, those bridges being closest to the center of the first length P are rectilinear, whereas the bridges arranged proximal of the ends of the first length P comprise bends. This configuration allows to obtain more yielding bridges, as is well known in the stent field.

In accordance with another embodiment, the bridges are made of different materials. Particularly, those bridges being closest to the center of first length P are made of a harder material, whereas those bridges being arranged proximal of the ends of the first length P are made of a more elastic material.

With reference to FIGS. 11, 11a and 12, with 320 there is indicated a second embodiment of a stent according to the invention as a whole.

The stent 320 comprises a first cylindrical portion 321 and a second cylindrical portion 322. The cross-section of the cylindrical portions 321 and 322, in accordance with the geometric definition of cylinder in the broadest meaning of the word, can be circular, elliptical or any other loop which may be suitable to the particular use of the stent 320.

The first portion 321 and the second portion 322 are structurally connected to each other by means of at least two bridges 323. The bridges 323 engage a first length along the closed loops being defined by the cross section of the stent. For example, the first length of the closed loop defined by the first portion 321, which is engaged by the bridges 323, is indicated with P in FIG. 11a. A second length of the closed loop is thus also defined, which is a complementary length to the first one and is not interested by the presence of the bridges 323. This second length is indicated with S in FIG. 11a.

In the stent 320 according to the invention, the first length P is shorter than second length S. In other words, the structural connection between both portions 321 and 322 of the stent 320 comprises a plurality of bridges 323 unevenly distributed over the closed loop being defined by the cross section of the portions.

In accordance with one embodiment, the first length P is shorter than half, preferably shorter than one third, and still more preferably one fourth of second length S.

The y-y axis of the first portion 321 and the z-z axis of the second portion 322 are different. Furthermore, the closed loops being defined by the cross sections of the first portions 311 and second portions 312 are disjoint and have only a part of the perimeter in common. In other words, the proximal end of first portion 321 is connected by means of the bridges 323 to the proximal end of second portion 322.

In accordance with an embodiment, the bridges 323 have different lengths and/or elasticities. Those bridges being closest to the center of first length P of the closed loop are required be shorter and/or less elastic than the bridges arranged proximal of the ends of the first length P.

With reference to FIGS. 13, 13a and 14, with 330 is indicated a third embodiment of a stent according to the invention as a whole.

The stent 330 comprises both the characteristics of the first 310 and second 320 embodiments of the stent according to the invention.

The stent 330 comprises a first cylindrical portion 331, a second cylindrical portion 332 and a third cylindrical portion 334. The cross section of the cylindrical portions 331, 332 and 334, in accordance with the geometric definition of cylinder in the broadest meaning of the word, can be circular, elliptical, or any other closed loop which may be suitable to the particular use of the stent 330.

The first portion 331 is structurally connected to the second portion 332 and the third portion 334 by means of at least two bridges 333. Similarly to what has been described above, the bridges 333 engage along the closed loops defined by the cross section of the stent a first length p shorter than the second length S which is complementary to the first one and is not interested by the presence of the bridges 313 (the lengths P and S are not represented in FIG. 13a for clarity reasons). In other words, the structural connection between the portions 331 and 332 and the portions 331 and 334 of the stent 330 comprises a plurality of bridges 333 unevenly distributed along the closed loop being defined by the cross section of the portions.

In accordance with an embodiment, the first length P is shorter than half, preferably shorter than one third, and still more preferably shorter than one fourth of second length S.

Furthermore, in accordance with a preferred embodiment, the y-y axis of the first portion 331, the z-z axis of the second portion 332 and the w-w axis of the third portion 334 are different. Furthermore, the closed loops being defined by the cross sections of the first portion 331 and third portion 334 are at least partially comprised in the closed loop being defined by the cross section of second portion 332. Likewise, the closed loops being defined by the cross sections of the first portion 331 and third portion 334 are disjoint and have only a part of their perimeter in common. In other words, the proximal end of the first portion 331 is connected to the distal end of the second portion 332, and the proximal end of the third portion 334 by means of the bridges 333.

In accordance with an embodiment, the bridges 313 have different lengths and/or elasticities. In fact, those bridges that are closest to the center of the first length P of the closed loop are required to be shorter and/or less elastic than the bridges being arranged proximal of the ends of the first length P.

With reference to FIG. 6, with 400 there is indicated an assembly for the treatment of stenosis according to the invention. The assembly 400 comprises a first catheter 100 and a stent 310, 320 or 330 in accordance with what has been described above. The assembly further comprises a mandrel 180, preferably hollow, which is placed beside catheter 100. The balloon 130 of catheter 100 is in its collapsed condition, being folded about the tubular body 110 of catheter 100. The first portion of the stent 311, 321 or 331 is fitted on the balloon 130. On the other hand, the second portion 312 or 332 is simultaneously fitted on the balloon 130 and the mandrel 180. The second portion 322 or the third portion 334 are fitted only on the mandrel 180.

In accordance with a preferred embodiment, the mandrel 180 of assembly 400 also passes through the eyelet 150.

With reference to FIG. 7 with 500 there is indicated an assembly for the treatment of stenosis according to the invention. The assembly 500 comprises a first catheter 100 and a stent 310, 320 or 330 in accordance with what has been described above. The assembly further comprises a second catheter 200 being placed beside first catheter 100. Both balloons 130 and 230 of both catheters 100 and 200 are in their collapsed condition, being folded about the tubular bodies 110 and 210 of the catheters. The first portion of the stent 311, 321 or 331 is fitted on the balloon 130 of first catheter 100. On the other hand, the second portion 312 or 332 is simultaneously fitted on the balloon 130 of first catheter 100 and the balloon 230 of second catheter 200. The second portion 322 or the third portion 334 are instead fitted only on the balloon 230 of second catheter 200.

The second catheter 200 of assembly 500 also passes through the eyelet 150 of first catheter.

The method for preparing the assembly 500 generally provides that, starting from the assembly 400, the mandrel 180 is removed from the stent 310 or 320 or 330 and from the eyelet 150, if required. The second stent portion 312 or 322 or 332, and the third stent portion 334, if provided, form a slot which is partially engaged by the balloon 130 of the first catheter 100 and partially free.

After the mandrel 180 has been removed, the second catheter 200 has to be selected based on the particular conditions of use. The distal end 220 of second catheter 200 is then inserted in the eyelet 150 and advanced until reaching the partially free slot being formed by the stent 310 or 320 or 330.

The distal end 220 is then inserted in the slot being formed by the stent 310 or 320 or 330. The second catheter 200 is then advanced over the first catheter 200 until the shoulder 250 abuts against the eyelet 150. Because the shoulder outer diameter and the eyelet inner diameter are such that the shoulder abuts against the eyelet without being able to pass therethrough, an end of stroke is thereby formed univocally defining a mutual positioning of both catheters 100 and 200, and particularly of both balloons 130 and 230.

In accordance with an embodiment, the method described above provides that the mandrel 180 be hollow. According to this embodiment of the method, before removing the mandrel 180 from the stent 310 or 320 or 330 and from the eyelet 150, if necessary, a stylet or guide wire 185 is inserted in the mandrel 180 and the eyelet 150. Thereby, after the mandrel has been removed and the second catheter 200 has been fitted on the stylet or guide wire 185, inserting the second catheter 200 in the eyelet and slot being formed by the stent is easier.

In accordance with an embodiment, upon completion of this insertion, the stylet or guide wire 185 is removed thus obtaining the assembly 500 described above.

The method for using the assembly 500 according to the invention provides that the operator inserts, in a manner known per se, a couple of guide wires along the patient's vessels such as to reach the bifurcation as desired. A first guide wire is placed within the side branch of the bifurcation, whereas the second guide wire is placed within the main branch.

After the catheters and the stent have been selected according to the particular requirements and after the assembly 500 has been prepared according to what has been stated above, the operator inserts the first catheter 100 on the first guide wire and the second catheter 200 on the second guide wire.

The particular case of the first embodiment 310 of the stent will be considered below, but the description of this method likewise applies to the subsequent embodiments 320 and 330 of the stent according to the invention.

The catheters, by being advanced by the operator over the guide wires, almost automatically reach the position shown in FIG. 8. Due to the interaction between the shoulder 250 of second catheter 200 and the eyelet 150 of first catheter 100, the relative position of both balloons is univocally defined and maintained under the effect of the thrust applied by the operator. In FIG. 8 there is illustrated a position where the proximal ends of the balloons are substantially aligned, but with a different arrangement of the eyelet 150 and shoulder 250 along the respective catheters, it is possible to determine other positions that may be particularly useful in several specific situations.

In accordance with an embodiment of the assembly, for example, the proximal end of the balloon 230 of second catheter 200 is arranged at a certain distance in the proximal direction relative to the proximal end of the first balloon 130, such that the proximal end of the first balloon 130 is distally placed relative to the proximal end of the second balloon 230, as in the example from FIG. 4.

With this particular arrangement of both balloons being slightly offset in the axial direction, an improved inner profile can be provided to the stent and accordingly to the bifurcation treated by the angioplasty.

After the assembly 500 has been moved to the position illustrated in FIG. 8, the operator brings the balloons from the collapsed condition to the expanded condition.

In accordance with an embodiment of the method the inflation of both balloons 130 and 230 is carried out at the same time.

In accordance with another embodiment of the method, the first balloon 130 is inflated prior to second balloon 230.

In a manner known per se, the first balloon 130 dilates the stenosis and restores the vessel inner diameter, which is thereby brought back to non-pathological values.

By inflating the first balloon 130 the first portion 311 of stent 310 is also dilated and brought from its collapsed condition to its expanded condition. In the expanded condition, the first section 311 supports the inner walls of the side branch to avoid that, after the angioplasty operation, they may shrink and reduce the inner diameter back to pathological levels.

By inflating the balloons 130 and 230, the second portion 312 of the stent 310 is also dilated and is brought to its collapsed condition to its expanded condition. In the expanded condition the second section 312 supports the area immediately proximal of the side branch, indicated with A in FIG. 1a. As can be clearly seen in FIG. 10, after both catheters 100 and 200 have been removed, the stent 310 once definitely placed, is totally adhered to the bifurcation walls and does not interferes at all with the blood stream. For this reason, if the bifurcation is affected by stenosis even along the main branch, the stent 310 according to the invention can be used by being coupled with a stent b of the known type 600 being dedicated to the main branch of a bifurcation. A situation of this type is shown in FIG. 15.

As refers to the specific characteristics of the second embodiment 320 of the stent, it is particularly suitable for the treatment of the carina area, which is indicated with B in FIG. 1b. Due to the presence of both cavi guida and the particular structure of the assembly 500 according to the invention, the operator can place the stent 320 in the proper position in an almost automatical manner. In fact, the first catheter 100, following its guide wire, reaches the side branch and thus the first portion 321 of the stent 320. The second catheter 200, following its guide wire, reaches the main length and the second portion 322 of the stent 320. Under the thrust action by the operator, the bridges 323 connecting both portions of the stent abut against the cusp of the carina (see for example FIG. 12) thus univocally defining the position of the stent in the bifurcation.

As refers to the specific characteristics of the third embodiment 330 of the stent, it is particularly suitable for treating both the more proximal area of the bifurcation, being indicated with A in FIG. 1a, and the area of the carina, being indicated with B in FIG. 1b. In fact, the third embodiment comprises the characteristics of the first and second embodiments described above.

In accordance with an embodiment of the method for preparing the assembly 500, the operator inserts a first guide wire along the patient's blood vessels until it is arranged in the side branch of the bifurcation in question. The guide wire 85 described above and inserted in the hollow mandrel 180 acts as the second guide wire, being arranged in the main branch of the bifurcation. After the mandrel 180 has been removed, the first catheter 100 is advanced over the first guide wire until reaching the bifurcation. Only at this time the second catheter 200 is fitted on the second guide wire 185 and advanced thereonto until reaching the bifurcation. The second catheter will be automatically inserted in the eyelet 150 and the slot formed by the stent, such as to form the assembly 500.

The embodiments of the method described above for preparing the assembly 500 do not substantially differ from the latter. The only difference is that with this latter embodiment of the method, the second catheter 200 will reach its operative position, i.e. with the shoulder 250 abutting against the eyelet 150, when it is already inserted in the patient's body. Thus, this embodiment of the method is advantageous in that the catheters run through the patient's vessels separately, the bifurcation interested by the operation being thereby easier to reach. On the other hand, the embodiments of the method described above are advantageous in that they can be carried out at a separate time than angioplasty operation. For example, the preparation of the assembly 500 from the assembly 400 and second catheter 200 can take place, once the size of the main branch and side branch of bifurcation are acknowledged, in a laboratory remote from the operation site.

To the above embodiments of the stent, catheters, assemblies and methods thereof, those skilled in the art, aiming at satisfying contingent requirements, may be able to carry out modifications, adaptations and replacements of elements with others being functionally equivalent, without departing from the scope of the claims below. Each of the characteristics being described as belonging to a possible embodiment can be carried out independently of the other embodiment described.

1. An endoluminal stent comprising:
at least one first cylindrical portion having an axis and an annular section, said annular section comprising a first length and a second length, and
at least one second cylindrical portion having an axis and being connected to said first cylindrical portion,
wherein the connection between said first and second cylindrical portions comprises at least two bridges and engages said first length of said annular section of said first cylindrical portion, said first length being shorter than said second length, and
wherein said axis of said first cylindrical portion and said axis of said second cylindrical portion are different.

2. The stent according to claim 1 comprising:
a third cylindrical portion having an axis and being connected to said first cylindrical portion,
wherein the connection between said first and third cylindrical portions comprises at least two bridges and engages said first length of said annular section of said first cylindrical portion, said first length being shorter than said second length, and
wherein said axis of said first cylindrical portion and said axis of said third cylindrical portion are different.

3. The stent according to claim 2, wherein said axis of said second cylindrical portion and said axis of said third cylindrical portion are different.

4. The stent according to claim 1 wherein said connection is unevenly arranged along the annular section of said first cylindrical portion.

5. The stent according to claim 1 wherein said first length is shorter than half said second length.

6. The stent according to claim 1 wherein said first length is shorter than one third of said second length.

7. The stent according to claim 1 wherein said first length is shorter than one fourth of said second length.

8. The stent according to claim 1 wherein said bridges have different lengths.

9. The stent according to claim 1 wherein said bridges are made of different materials.

10. The stent according to claim 1 wherein said bridges have different elasticities.

11. A catheter for endoluminal operations comprising a main tubular body having a distal end, a proximal end and a proximal port for a guide wire, said distal end comprising a balloon and a distal port for a guide wire, wherein an eyelet is placed at a preset distance in the proximal direction relative to said balloon.

12. The catheter according to claim 11 wherein said eyelet is placed laterally relative to said tubular body.

13. The catheter according to claim 11 wherein said eyelet is integral with said tubular body.

14. The catheter according to claim 11 wherein said eyelet defines an x-x axis being locally parallel to said tubular body.

15. The catheter according to claim 11 wherein said eyelet comprises a slot of substantially unextensible thread.

16. The catheter according to claim 11 wherein said eyelet comprises a tube length being substantially fastened to said tubular body.

17. The catheter according to claim 16 wherein said tube length is fastened to said tubular body by welding.

18. The catheter according to claim 16 wherein said tube length is fastened to said tubular body by means of gluing.

19. The catheter according to claim 16 wherein said length of tube and said tubular body are made as one piece by extrusion.

20. The catheter according to claim 11 comprising a sheath simultaneously enveloping the tubular body and the eyelet.

21. The catheter according to claim 16 wherein said tube length is cut along a plane perpendicular to the x-x axis.

22. The catheter according to claim 16 wherein said tube length is cut along a curved and biased surface relative to x-x axis.

23. The catheter according to claim 11 wherein said proximal port for the guide wire is proximally located relative to said eyelet.

24. A catheter for endoluminal interventions comprising a main tubular body having a distal end, a proximal end and a proximal port for a guide wire, said distal end comprising a distal port for a guide wire and a balloon suitable to be inflated,
wherein a shoulder is placed at a preset distance in the proximal direction relative to said distal end.

25. The catheter according to claim 24 wherein said distal end, when said balloon is not inflated, is suitable to be inserted in the eyelet of a catheter comprising a main tubular body having a distal end, a proximal end and a proximal port for a guide wire, said distal end comprising a balloon and a distal port for a guide wire, wherein an eyelet is placed at a preset distance in the proximal direction relative to said balloon, wherein said shoulder is not suitable to be inserted in the eyelet of said catheter.

26. The catheter according to claim 24 wherein said shoulder comprises a step perpendicular to the outer surface of said tubular body.

27. The catheter according to claim 24 wherein said shoulder comprises a tapering joining the shoulder maximum diameter to the diameter of said tubular body.

28. An assembly comprising
a first catheter according to claim 11,
a mandrel and
a stent comprising at least one first cylindrical portion having an axis and an annular section, said annular section comprising a first length and a second length, and at least one second cylindrical portion having an axis and being connected to said first cylindrical portion, wherein the connection between said first and second cylindrical portions comprises at least two bridges and engages said first length of said annular section of said first cylindrical portion, said first length being shorter than said second length, and wherein said axis of said first cylindrical portion and said axis of said second cylindrical portion are different.

29. The assembly according to claim 28 wherein said first and second stent portions are fitted on said first catheter and wherein said second portion of said stent is fitted on said mandrel.

30. The assembly according to claim 28 wherein said first portion of said stent is fitted on said first catheter and wherein said second portion of said stent is fitted on said mandrel.

31. The assembly according to claim 28 wherein said first and second stent portions are fitted on said first catheter and wherein said second and third portions of said stent are fitted on said mandrel.

32. An assembly comprising
a first catheter according to claim 11,
a second catheter comprising a main tubular body having a distal end, a proximal end and a proximal port for a guide wire, said distal end comprising a distal port for a guide wire and a balloon suitable to be inflated, wherein a shoulder is placed at a preset distance in the proximal direction relative to said distal end and
a stent comprising at least one first cylindrical portion having an axis and an annular section, said annular section comprising a first length and a second length, and at least one second cylindrical portion having an axis and being connected to said first cylindrical portion, wherein the connection between said first and second cylindrical portions comprises at least two bridges and engages said first length of said annular section of said first cylindrical portion, said first length being shorter than said second length, and wherein said axis of said first cylindrical portion and said axis of said second cylindrical portion are different.

33. The assembly according to claim 32 wherein said first and said second stent portions are fitted on said first catheter and wherein said second portion of said stent is fitted on said second catheter.

34. The assembly according to claim 32 wherein said first portion of said stent is fitted on said first catheter and wherein said second portion of said stent is fitted on said second catheter.

35. The assembly according to claim 32 wherein said first and said second portions of said stent are fitted on said first catheter and wherein said second and said third portions of said stent are fitted on said second catheter.

36. A method for preparing an assembly of a stent, a first catheter and a second catheter, said method comprising steps of:
arranging an assembly according to claim 28;
arranging said second catheter;
removing said mandrel such that at least a portion of said stent is left free;
inserting the distal end of said second catheter in said eyelet of said first catheter;
inserting the distal end of said second catheter in said at least one partially free portion of said stent;
advancing said second catheter in the distal direction along said first catheter until said shoulder comes in contact with said eyelet.

37. The method according to claim 36, wherein before removing said mandrel, a stylet or guide wire is inserted in said mandrel and said eyelet and the distal end of said second catheter is fitted on said stylet or guide wire.

38. A method for using an assembly according to claim 32 in a bifurcation of a blood vessel being affected by stenosis wherein said method is characterized by the following steps:
arranging a first guide wire in a side branch of a bifurcation;
arranging a second guide wire in a main branch of a bifurcation;
fitting said first catheter on said first guide wire;
fitting said second catheter on said second guide wire;
advancing both catheters simultaneously along both guide wires until said balloons reach said bifurcation;
inflating said balloons;
deflating said balloons;
removing said second catheter;
removing said first catheter.

39. The method according to claim 38 wherein said balloons are inflated at the same time.

40. The method according to claim 38 wherein said first balloon is inflated before said second balloon.

41. A method for employing an assembly according to claim 37 in a bifurcation of a blood vessel being affected by stenosis wherein said method is characterized by the following steps:
arranging a first guide wire in a side branch of a bifurcation;
arranging said guide wire in a main branch of a bifurcation;
fitting said first catheter on said first guide wire;
fitting said second catheter on said guide wire;
advancing said first catheter over said first guide wire until said balloon reaches said side branch of said bifurcation;
advancing said second catheter along said guide wire until said shoulder comes in abutment against said eyelet;
inflating said first balloon;
deflating said second balloon;
deflating said balloons;
removing said second catheter;
removing said first catheter.

42. The method according to claim 41 wherein said balloons are inflated at the same time.

43. The method according to claim 41 wherein said first balloon is inflated before said second balloon.

44. The method for employing an assembly according to claim 37 in a bifurcation of a blood vessel affected by stenosis wherein said method is characterized by the following steps:
arranging a first guide wire in a side branch of a bifurcation;
arranging said guide wire in a main branch of a bifurcation;
fitting said first catheter on said first guide wire;
fitting said second catheter on said guide wire;
advancing said first catheter along said first guide wire until said balloon reaches said side branch of said bifurcation;
inflating said first balloon;
deflating said first balloon;
advancing said second catheter along said guide wire until said shoulder comes to abutment against said eyelet;
inflating said second balloon;
deflating said second balloon;
removing said second catheter;
removing said first catheter.
